(19) United States
(12) Patent Application Publication
(10) Pub. No.: US 2004/0092810 A1
(43) Pub. Date: May 13, 2004

(54) Method and apparatus for MR-guided biopsy

(76) Inventors: Wolfgang Daum, Groton, MA (US); Axel Winkel, Schwerin (DE)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/366,831

(22) Filed: Feb. 14, 2003

Related U.S. Application Data
(60) Provisional application No. 60/357,205, filed on Feb. 14, 2002.

(51) Int. Cl. A61B 5/05

(52) U.S. Cl. 600/411

(57) ABSTRACT

The subject invention pertains to a method and apparatus for MR-guided biopsy. The subject invention can be applied to, for example, prostate biopsy. In a specific embodiment, the subject invention can provide a mechanical tool for stabilizing the patient in prone position and to guide a biopsy needle into defined targeted lesions in the prostate gland. The patient can lay prone in the MRI. The subject apparatus can guide an MR-visible, sterile needle sleeve, which can have a hollow tube filled with contrast media, through the anus onto the inner wall of the colon. Due to the visibility of the contrast media in the sleeve, the apparatus can be guided to the exact position. The sleeve can incorporate a tube within the contrast media filled sleeve to insert the biopsy needle and to push this needle forward into the prostate. The subject apparatus can utilize various mechanical means to stereotactically move the needle or needle sleeve in various directions.

### CROSS-REFERENCE TO RELATED APPLICATION

The present application claims the benefit of U.S. Provisional Application No. 60/357,205, filed Feb. 14, 2002, which is hereby incorporated by reference herein in its entirety, including any figures, tables, or drawings.

### FIELD OF INVENTION

The subject invention pertains to a method and apparatus for MR-guided biopsy. The subject invention can be applied to prostate biopsy. In a specific embodiment, the subject invention relates to a stereotactic positioning device for MR-guided interventions, such as biopsies of suspicious areas of the prostate gland. MRI (magnetic resonance imaging) is a current radiological imaging modality to view soft tissue lesions of the human body. MR can be used to guide the subject positioning device to directly puncture lesions in the prostate and/or to biopsy these.

### BRIEF SUMMARY OF INVENTION

The subject invention pertains to a method and apparatus for MR-guided biopsy. The subject invention can be applied to, for example, prostate biopsy. In a specific embodiment, the subject invention can provide a mechanical tool for stabilizing the patient in prone position and to guide a biopsy needle into defined targeted lesions in the prostate gland. The patient can lay prone in the MRI. The subject apparatus can guide an MR-visible, sterile needle sleeve, which can have a hollow tube filled with contrast media, through the anus onto the inner wall of the colon. Due to the visibility of the contrast media in the sleeve, the apparatus can be guided to the exact position. The sleeve can incorporate a tube within the contrast media filled sleeve to insert the biopsy needle and to push this needle forward into the prostate. The subject apparatus can utilize various mechanical means to stereotactically move the needle or needle sleeve in various directions.

### BACKGROUND OF THE INVENTION

Prostate cancer is the most common cancer, excluding skin cancers, in American men. The American Cancer Society estimates that during 2002 about 189,000 new cases of prostate cancer will be diagnosed in the United States. Accurate determination of the extent of local disease in the prostate is difficult. Current imaging techniques include, for example, transrectal ultrasound (TRUS), endorectal coil magnetic resonance imaging (MRI), and proton magnetic resonance spectroscopic imaging (MRSI). The reported accuracy of TRUS for determining if prostate cancer is confined within the capsule varies widely from 58% to 90%. However, preliminary data from recent studies of endorectal MRI show higher accuracy (75-90%) than TRUS, and better consistency.

In addition to morphologic extent, directed biopsy and assessment of tumor aggressiveness are important for accurate staging and treatment for prostate cancer when there is an elevated PSA. Current biopsy techniques are based on random spatial sampling and have a lower than desired sensitivity (60-70%) for identification of carcinoma of the prostate. Early preliminary studies of combined MRI/MRSI demonstrated localization of cancer to a sextant of the prostate with sensitivity up to 95% and specificity up to 91%. However, more specifically localized biopsies, rather than randomly taken biopsies, would be desirable.

MRI is presently regarded as the best imaging modality for assessing soft-tissue tumors like prostate cancer. This is confirmed by numerous reports in the literature. In an early study, carried out from December 1987 to April 1989, Rifkin et al [7] report on the collaborative effort of five institutions that are part of the Radiological Diagnostic Oncology Group. More than 200 patients who were thought clinically to have localized cancer of the prostate were studied preoperatively with both MRI and transrectal ultrasonography to evaluate the ability of these techniques to determine the exterit (stage) of the tumor. They underwent radical prostatectomy, and radiologic and pathological findings were correlated. The overall staging accuracy of ultrasonography was 58% (126 of 219 patients), with a standard error of 3%. The overall staging accuracy of MRI was 69% (133 of 194 patients), with a standard error of 3%. The subject invention can increase the diagnostic accuracy of MRI when combining MRI scans with interventional biopsy techniques.

Prostate cancer is the second most common cause of cancer death in US men. Its incidence is on the rise because more cancers are detected due to wide-ranging screening programs using either digital rectal exams or serum prostate-specific antigen (PSA). Whenever abnormalities crop up in these examinations, the patient is traditionally referred for ultrasound-guided biopsy, which has a low sensitivity and a specificity of only 60% for cancer detection [3]. This is why ultrasound is often used just to guide biopsies. However, MRI performs much better at cancer detection.

Typical prostate biopsies are performed by palpation (whether or not a nodule is present) or using ultrasound guidance (when a visible lesion is present). However, endorectal ultrasound is not sensitive enough for a screening tool. The visibility of the anterior capsule is poor as is visualization of seminal vesical and lymph node involvement. Extracapsular disease and lymph node involvement is better picked up with MR, although interobserver variability is quite high (positive predictive value ˜70%). PSA and proton MR spectroscopy get higher ratings for predicting the Gleason grade. Patients with incompatible PSA and biopsy results or MR spectroscopy results or with MR visible lesions would thus benefit from an MR guided prostate biopsy.

### BRIEF DESCRIPTION OF THE DRAWINGS

The invention is explained in the following figures:

FIG. 1 illustrates a prostate biopsy system in accordance with the subject invention, patient lying prone.

FIG. 2 illustrates a stick to remotely operate an embodiment of the subject device from outside the MR magnet.

FIG. 3a illustrates a three dimensional view of an embodiment of the subject invention, from a more frontal point of view.

FIG. 3b illustrates a three dimensional view of an embodiment of the subject invention, from a more back point of view.

FIG. 4a illustrates a cross-sectional view of disposable needle-sleeve and needle-sleeve-block in accordance with the subject invention.

FIG. 4b illustrates a super-side view of a disposable needle-sleeve and needle-sleeve-block in accordance with the subject invention.

FIG. 4c illustrates a lock-in mechanism of a disposable needle block in accordance with the subject invention.

FIG. 5a illustrates a disposable biopsy needle which can be used with an embodiment of the subject invention.

FIG. 5b illustrates a needle guide with depth control which can be incorporated with an embodiment of the subject invention

FIG. 6 illustrates a cross-sectional view of a straight biopsy device in accordance with the subject invention, which is attached to a patient's body.

FIG. 7 illustrates a cross-sectional view of a curved biopsy device in accordance with the subject invention, which is attached to the patient's body.

FIG. 8 illustrates the different biopsy locations in the prostate gland.

FIG. 9 illustrates a three dimensional view of another specific embodiment of the subject invention.

Numbers used to describe the features in the drawings:

1 patient

2 biopsy system

3 anus

4 positioning cushion

5 prostate

6 holding arm

7 axis

8 needle holder

9 stick

10 grip

11 tool to attach to parts of the prostate biopsy system

12 grip of the biopsy needle 2

13 positioning cushion

14 base plate

15 arm-mounting-track

16 lower arm

17 upper arm

18 arm-mounting-track

19 adjustment screw

20 sliding-part of the lower arm 16

21 base-part of the lower arm 16

22 needle-sleeve holder

23 lock-bolt

24 needle-sleeve

25 needle-sleeve-block

26 needle-sleeve-lock-in mechanism

27 outer tube

28 inner tube

29 seal stop

30 hollow space

31 snap-on

32 positioning system

33 patient

34 ball-and-socket-joint

35 curved inner needle

36 outer straight needle

37 grip of 36

38 grip of 35

39 prostate

40 ureter

41 mechanical axial fixation

42 locking mechanism

43 locking lever

44 mechanical fixation

45 coaxial hub tube

46 sliding stopper

47 needle plate

48 biopsy device

49 lock mechanism

### DETAILED DESCRIPTION OF THE INVENTION

The subject invention pertains to a method and apparatus for MR-guided biopsy. In a specific embodiment, the subject invention can be utilized for prostate biopsy. In a specific embodiment, the subject invention relates to a positioning device for prostate interventions, which can incorporate many parts, such as a biopsy needle, a needle sleeve, various positioning and adjustment parts, coils, and more.

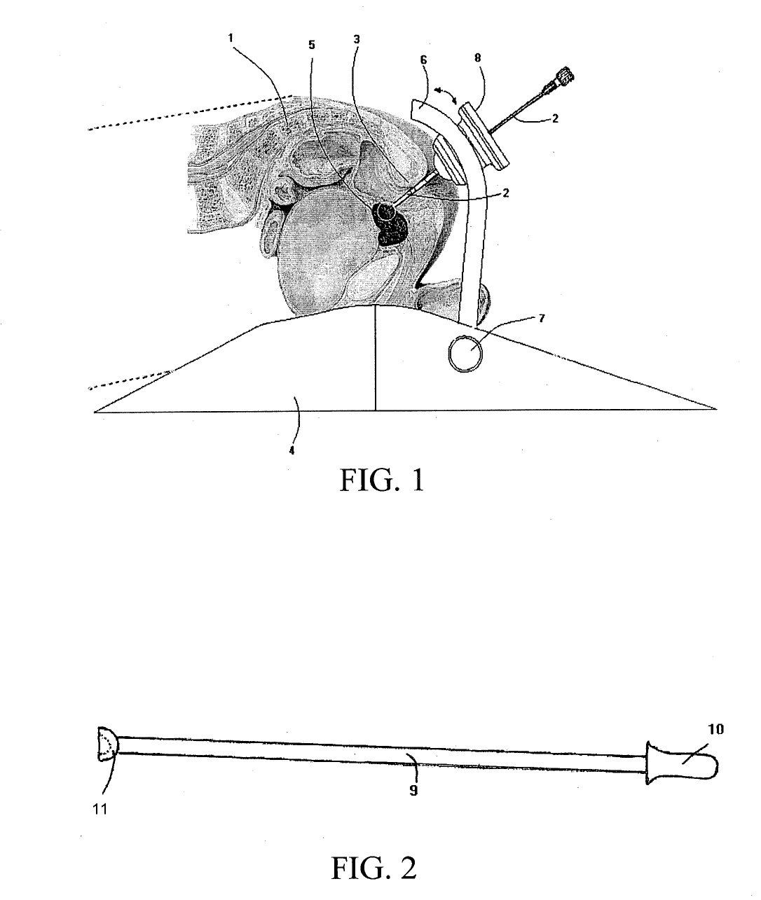

FIG. 1 illustrates a specific embodiment of the subject prostate biopsy system in use with a patient. The patient 1 is lying in a prone position and the biopsy system 2 is introduced endorectally through the anus 3. A specially shaped positioning device 4 is positioned under the patient's hips to stabilize the patient as needed for the procedure. Positioning device 4 can provide cushion to the patient. In this position the patient's back side is lifted up a little, so that the physician has better access through the patient's anus 3. In FIG. 1 the biopsy needle 2 is directly inserted through the anus 3 of the patient through the intestine wall of the rectum directly into the prostate. Here no special introducer device, as described is used. On the positioning device 4 is mounted a holding arm 6, which is movable around an axis 7 and is adjustable in height. Attached to the holding arm 6 is the needle holder 8 through which the biopsy needle 2 will slide.

There are two ways to operate this embodiment, and later described prostate biopsy embodiments, in conjunction with magnetic resonance imaging (MRI). In a first technique the patient is pulled out the MR magnet to operate the device, pushed back in the magnet to control the position of the needle guide, and pulled out the magnet for further needle adjustments if needed. The dimensions for the necessary corrections can be taken from the image and transferred to the scales of the device. In a second technique, the patient stays in the magnet and images are taken during the needle repositioning procedure. The device will appear in the image and is operated from the outside by simply reaching in with the arm of the operator or by remotely operated tools. These tools can be, for example, long plastic sticks 9. Sticks 9 can be between 50 cm and 150 cm long, and 5 mm to 20 mm in diameter. Sticks 9 can have a grip 10 on the proximal end and a tool 11 at the distal end to attach to a particular part of the prostate biopsy device. The attachment tool 11 can, for instance, attach directly to the grip 12 of the biopsy needle 2 for the purpose of adjusting the position and pushing the needle 2 into the tissue. The attachment tool 11 can be changed to attach to different parts. The stick 9 can be extended in length during the operation or there can be sticks of various defined preset lengths.

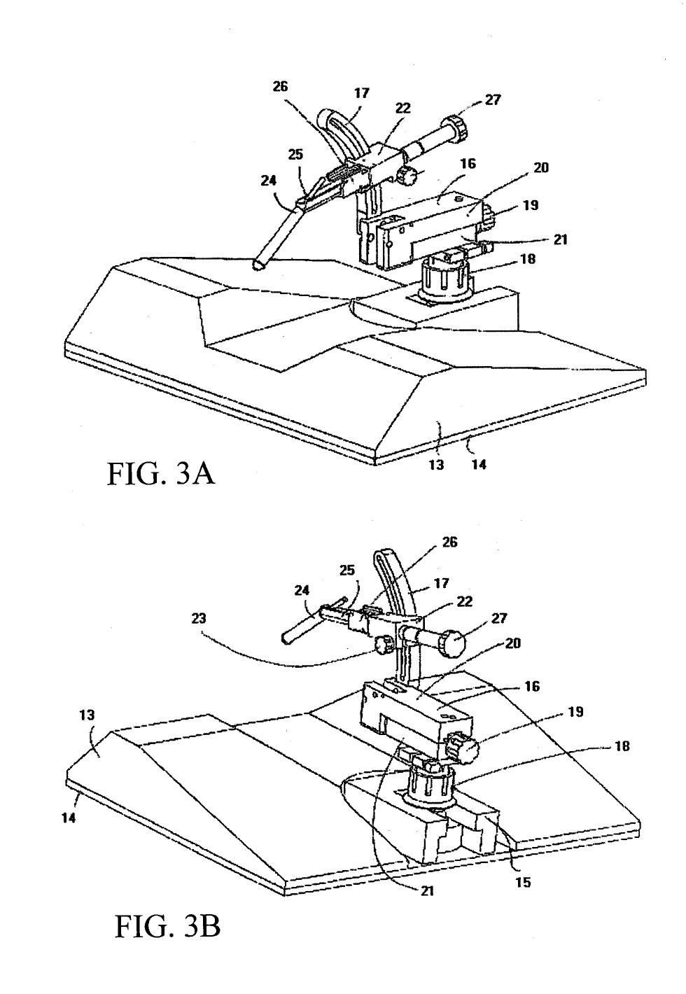

FIG. 3 shows another embodiment of the subject invention. FIG. 3A shows the device from a frontal view, while FIG. 3B shows the device from a rear view. Positioning device 13 is mounted on the base plate 14. Positioning device 13 can also provide cushion for the patient. The arm of the device, including lower arm 16 and upper arm 17, is locked in the arm-mounting-track 15 of the base plate 14 by arm-mounting-lock-bolt 18. Operating the adjustment-screw 19 allows the lower arm 16 to lengthen or shorten itself by means of a spiral-drive, not further shown here, within the lower arm 16. The spiral-drive moves the sliding-part 20 of the lower arm 16 against the fixed base-part 21 of the lower arm 16. The upper arm 17 is fixed on the distal end of the sliding-part 20 of the lower arm 16 and is designed to be a curved track for the needle-sleeve-holder 22. The needle-sleeve-holder 22 slides up and down the curved upper arm 17 and locks in the desired position via a locking mechanism, operated with lock-bolt 23. The curved upper arm 17 allows the movement of the needle-sleeve around a pivot point. In a specific embodiment, the pivot point is the anus 3, such that the patient can be positioned and the subject device adjusted so that as the needle-sleeve-holder 22 slides up and down the curved upper arm 17 the needle-sleeve moves about a pivot point, with the pivot point being the patient's anus. The needle sleeve 24 with needle-sleeve-block 25 can be a disposable device and can be changed via the needle-sleeve-lock-in mechanism 26, not further shown here. The needle sleeve 24 and needle-sleeve-block 25 can be moved forward and backwards via a spiral-drive mechanism, not further shown in detail here, by operating screw 27. This whole prostate-biopsy-device can be a reusable, and at least a cleanable, but most likely a sterilizeable unit. Screws, such as 19, 23 or 27, can be reached and operated with a stick 9, as shown in FIG. 2. Special adapting tools 11 are designed, but not further described here. In another embodiment of the invention, which is not further shown here, these screws are not manually operated, but motor operated with MR compatible motors such as piezo electric motors described in U.S. Pat. No. 6,274,965.

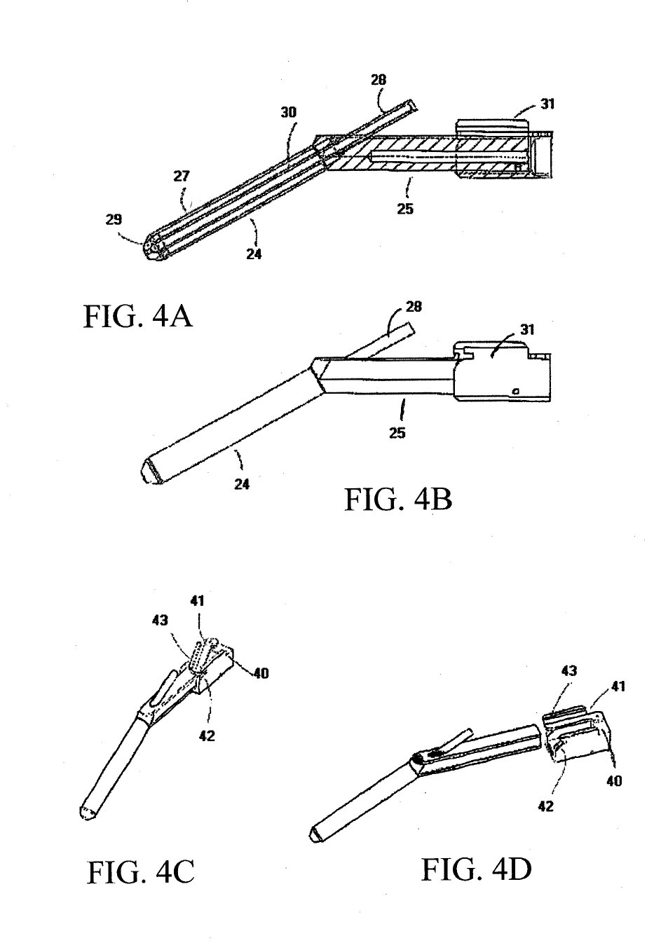

A specific embodiment of a needle-sleeve 24 and needle-sleeve-block 25 is shown in FIGS. 4A-4D. FIG. 4A illustrates the disposable needle-sleeve and needle-sleeve-block in cross sectional view and FIG. 4B illustrates a super side view of the disposable needle-sleeve and needle-sleeve-block. The needle-sleeve 24 incorporates an outer tube 27, which is sealed on its distal end by a seal-stop 29, or a moulded plastic ending, not further shown here. On the proximal side of the needle-sleeve 24 the needle-sleeve-block 25 seals the tube 27. An inner tube 28 penetrated through the entire length of the needle-sleeve 24. The hollow space 30 within the tube 27 is therefore sealed. This hollow space 30 can be filled with any contrast giving agent. In a specific embodiment, hollow space 30 can be filled with a MR positive contrast producing media with short T₁, T₂ or T₂* relaxation time. Examples of such media include Gd-DTPA (Gadolinium-diethylene-triaminepentacetic acid) and vitamin E. This contrast producing agent can allow the needle-sleeve 24 to be located easily under MR imaging. Very fast sequences can be used to show the needle guide. The section plan in which the biopsy should take place can be defined such that real time imaging in this plane can allow movement of the needle guide until it is perfectly lined up with the lesion. The needle guide can be fixed in this position and the biopsy can be taken outside the magnet.

The needle-sleeve-lock-in mechanism 26 allows a fast, safe and easy connection of the needle holder in the positioning device. Mechanical fixation 41 allows a precise lock-in in the longitudinal axis of the needle-sleeve-block 25. The mechanical fixation mechanism 42 has a squared cross section to prevent rotation of the needle-sleeve-block 25. The locking lever 43 fits into the mechanical fixation 41 at the opposite site.

The subject invention also relates to other techniques to make the needle sleeve visible for the MRI scanner. Fiducial markers, or other markers that use for example overhauser or electron spin can be incorporated. Two or three of this markers can exactly define the position of the needle sleeve and the way the needle will go. To save time it is possible to take a high resolution 3-D-image first and use the needle guide only to navigate. This has the advantage of fast nice pictures of the lesion in real time. For safety reason it might be desireable to take at least one image with the needle guide in place.

The biopsy needle can slide through inner tube 28, which can be aligned parallel to the outer tube 27. The inner diameter and length of tube 28 can match the outer diameter of the biopsy needle used. Typically the inner diameter is 8 to 16 G (gauge) or 1.7 to 3.0 mm. The needle-sleeve-block 25 with needle-sleeve 24 can be adapted to the needle-sleeve-holder 22 of the reusable prostate-biopsy system by, for example, a snap-on mechanism 31. For better orientation, the needle sleeve block can be filled with material which can produce contrast to show up in the image and indicate the axis of rotation of screw 27. In a specific embodiment, the needle sleeve can be made of materials substantially invisible to magnetic resonance imaging and a needle which is visible can be used.

The system incorporating the needle-sleeve 24 and it's sub-parts, the needle-sleeve-block 25, and the snap-on mechanism 31 can be made as one disposable part. This system can utilize plastic parts. Examples of plastic which can be utilized include but are not limited to, PE, PP, PU, PEEK or Teflon. Ceramic or low artifact giving metals, such as titanium and titanium-alloys can also be used.

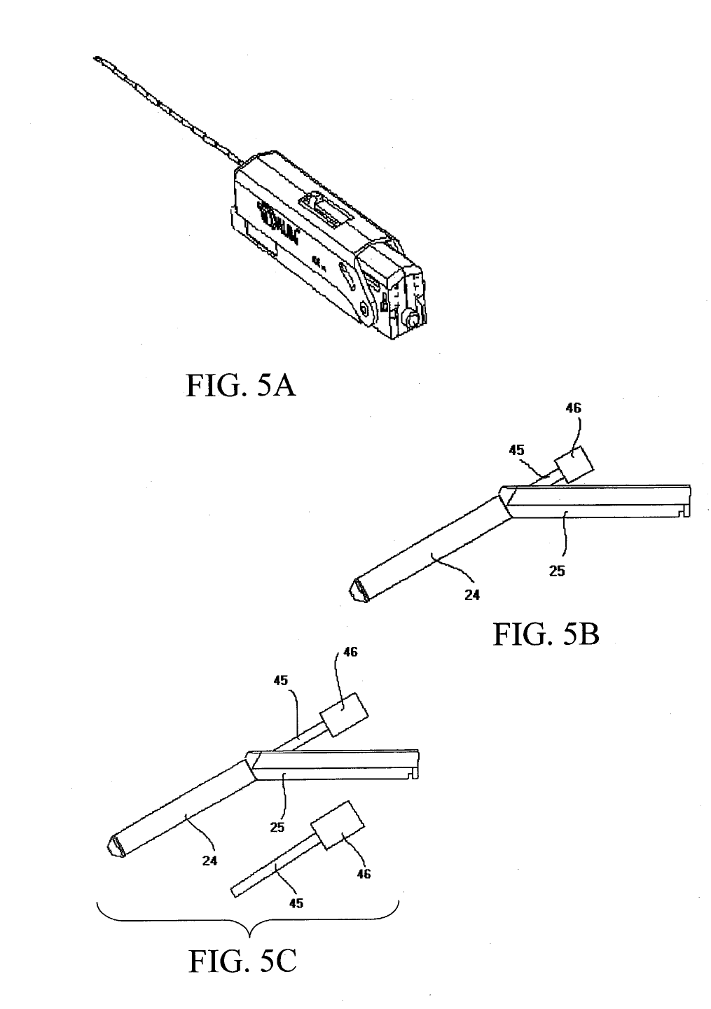

FIG. 5A shows a typical, disposable, fully automatic biopsy needle as used for this prostate biopsy device. The needle itself can be made out of a MR visible titanium alloy as described for instance in U.S. Pat. No. 6,120,517 or U.S. Pat. No. 5,895,401. Other surgical tools like the one in U.S. Pat. No. 6,238,355 can be inserted as well.

FIG. 5B shows a needle guide with depth control. The hub-tube 45 is coaxial and penetrates through the needle block 24 and has a stopper on it's proximal end. This hub-tube 45 shortens or lengthens the inner tube 28 of the needle block 24. Hence, if a needle, such as shown in FIG. 5a, penetrates through the inner-tube 28 it will have to stop at the stopper 46 and therefore can penetrate to a defined depth. This hub-tube 45 can be locked in position by a lock-in mechanism not shown herein.

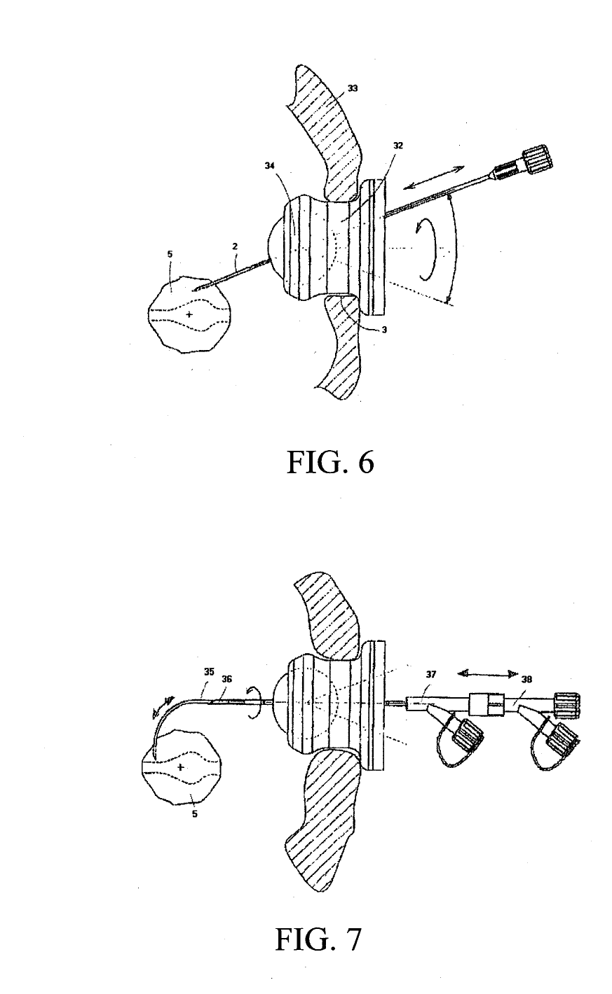

Another specific embodiment of the subject invention is shown in FIG. 6. The biopsy needle 2 is penetrating through the positioning system 32, which itself is only mounted to the patient 33 by clamping in the anus 3. The positioning system 32 comprises a ball-and-socket-joint 34, which allows a full angulated movement of the biopsy needle 2, as shown by curved arrows in FIG. 6. This positioning system 32 can be a disposable device, and can be made of MR compatible materials, such as PE, PP, PU, PEEK or Teflon. Ceramic or low artifact giving metals, such as titanium and titanium-alloys can also be used. FIG. 7 shows the same device with a needle 35, which is pre-bent and curves in a given direction when pushed out of a straight rigid needle 36. The curved needle 35 can be made out of, for example, super-elastic nickel-titanium (NiTi), the rigid and straight needle 36 can be made out of a titanium alloy, such as described in U.S. Pat. No. 6,238,355. Outer needle 36 is attached to grip 37, needle 35 is attached to grip 38. By grasping grip 38 with one hand and grip 37 with the other hand and pushing the one hand, and therefore grip 38, against the other hand, and therefore grip 37, needle 35 will be pushed out of needle 36 and will bend, as shown with the arrows.

FIG. 9 shows an alternative version of the prostate biopsy system. The needle plate 47 holds the biopsy device, which can be automated and driven by an MR compatible piezoelectric motor, for instance as shown in U.S. Pat. No. 6,274,965. This mechanism is posted on an upper arm 17, lower arm 16 and a base plate 14, all to be locked in defined positions by locking mechanism 49.

### EXAMPLE 1

This example describes a method for effecting a biopsy in accordance with the subject invention. In a specific embodiment, the subject prostate-biopsy-device can be operated in conjunction with a body faced array coil taking 6 to 8 samples, for example, by implementing the following:

Position the patient and the subject prostate-biopsy-device. Lay the patient prone on the stabilization pillow.

Install the body faced array coil, and the arm of the prostate biopsy device.

Insert the needle-sleeve through the anus onto the inner wall of the intestine posterior to the prostate (left apical corner of the prostate a).

Move the patient with device in the MR magnet and perform a first control scan (axial through prostate and needle sleeve).

Reposition the needle-sleeve if needed by moving the arm of the device from outside by using the sticks or move the patient out of the magnet and reposition manually the appropriate screws.

Measure the depth of the lesion in the prostate via another MR scan. If position is right move the patient out of magnet, introduce the biopsy needle through the needle sleeve into the prostate, and fire the biopsy needle to do the biopsy, or move the patient out of the magnet, introduce the biopsy through the needle sleeve into the prostate, fire the needle, and take a control image with the needle in place. Push out the needle notch, move the patient back into the MR magnet to make a controlling scan, and move the patient out of the magnet, to fire the biopsy needle to do the biopsy. (Or move the patient out of the magnet, introduce the biopsy through the needle sleeve into the prostate, drive the patient back into the MR magnet, and fire the biopsy needle to do the biopsy by using a stick from outside to operate the needle.) Alternatively, the hub-tube 45 of the device can be repositioned, so that the needle only penetrates to a certain depth.

Take out the first sample

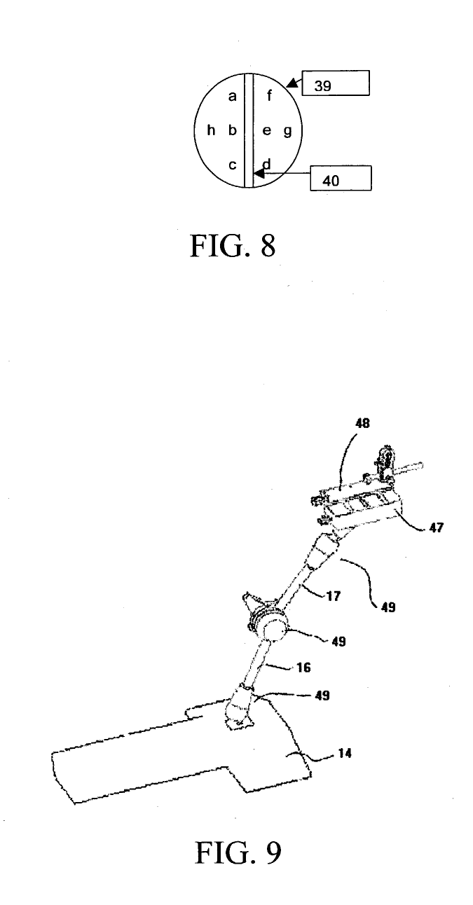

Move the sliding part of the lower arm 20 by turning adjustment screw 19 to position the needle sleeve in the middle b (referring to FIG. 8) of the left have of prostate and to the end c (referring to FIG. 8) of the prostate to take a biopsy from each position.

Position the needle guide at the right side of the prostate d (referring to FIG. 8) and take a control image. If the position is correct the patient is moved out of the magnet again and the next tree biopsies d, e, f (referring to FIG. 8) can be taken from the right side of the prostate in the same way as the left. For biopsies of special regions or additional lateral biopsies, the needle guide has to be positioned new and a control image has to be taken. The procedure described in this example allows a caregiver to take only two to four images to perform safe and fast biopsies with good control of the needle position. T2 weighted sequences can be used to view the prostate. After giving contrast media T1, weighted FLASH 3D sequences (SIEMENS 1.5T) can be used. For the intervention itself, a HASTE sequence or a T1 weighted can be used. For the can be used. In a 0.2 T SIEMENS MR tomographer, imaging was accomplished using a FLASH 2D-Sequence (TR/TE=100/9; 70 Grad), T2-SE (TR/TE=100/9; 70 Grad), and a FISP-Rotated-Keyhole-Sequence (TR/TE=18/8; 90 Grad).

1. A device for performing a prostate biopsy, comprising:
a needle sleeve for receiving a biopsy needle, wherein the needle sleeve is visible under magnetic resonance imaging; and
a means for holding the needle sleeve.
wherein the means for holding the needle sleeve allows an operator to position the needle sleeve in three dimensions.

2. The device according to claim 1, wherein the means for holding the needle sleeve is substantially invisible under magnetic resonance imaging.

3. The device according to claim 1, further comprising:
a means for positioning a patient, wherein the means for positioning a patient allows positioning of the patient with respect to the means for holding the needle sleeve.

4. The device according to claim 1, where the means for holding the needle sleeve allows an operator to position the needle sleeve while the operator is a sufficient distance from the needle sleeve such that the needle sleeve can be in an MR bore while the operator is outside of the MR bore.

5. The device according to claim 3, wherein the means for positioning the patient provides cushion to the patient

6. The device according to claim 1, wherein the means for holding the needle sleeve can be operated from outside the MR bore by remote control.

7. The device according to claim 6, wherein the means for holding the needle sleeve comprises ultrasound motors.

8. The device according to claim 6, wherein the means for holding the needle sleeve can be operated via one or more mechanical extension sticks.

9. The device according to claim 1, wherein the means for holding the needle sleeve allows the needle sleeve to be moved in all three dimensions at the same time.

10. The device according to claim 1, wherein the needle sleeve can be adjusted and clamped while being imaged under MRI.

11. The device according to claim 1, wherein the needle sleeve comprises material that provides a positive MR-signal.

12. The device according to claim 1, wherein the needle sleeve comprises one or more MR-visible markers.

13. The device according to claim 1, wherein the needle sleeve snaps on to be locked in.

14. The device according to claim 13, wherein the snap on mechanism has a positive fit for axial and transversal fixation and a mechanical lock for the third dimension.

15. The device according to claim 1, wherein the needle sleeve is adapted to accommodate different needle diameter and length.

16. The device according to claim 1, wherein the means for holding the needle sleeve is made of MR compatible materials.

17. The device according to claim 16, wherein the means for holding the needle sleeve is made of materials selected from the group consisting of: PE, PP, PU, PEEK, Teflon, ceramic, low artifact giving metals, titanium and titanium-alloys.

18. The device according to claim 3, wherein the patient lays prone.
